(19) United States
(12) Patent Application Publication
(10) Pub. No.: US 2011/0257097 A1
(43) Pub. Date: Oct. 20, 2011

(54) PHARMACEUTICAL COMPOSITIONS OF SOMATOTROPHIC HORMONES

(76) Inventors: Andrew Naylor, Nottingham (GB); Andrew Lester Lewis, Nottingham (GB); Lisbeth Illum, Nottingham (GB)

(21) Appl. No.: 13/003,515

(22) PCT Filed: Jul. 10, 2009
(86) PCT No.: PCT/GB2009/001727

§ 371 Date: Mar. 10, 2011

(30) Foreign Application Priority Data
Jul. 11, 2008 (GB) 0812740.9

(51) Int. Cl. A61K 38/27; A61P 3/04; A61P 13/12; A61P 5/06

(52) U.S. Cl. 514/11.3

(57) ABSTRACT

The invention provides a composition comprising (i) a somatotrophic hormone; (ii) a biodegradable polymer component; and (iii) a release modifier. A process for preparing, and the use of such a composition are also provided.

The invention relates to pharmaceutical compositions for the administration of somatotrophic hormones.

The listing or discussion of a prior-published document in this specification should not necessarily be taken as an acknowledgement that the document is part of the state of the art or is common general knowledge.

Somatotrophic hormones typically have to be administered by injection because they are inadequately absorbed by the body when they are administered by other routes. For example, patients requiring treatment by human growth hormone (hGH) currently are administered with a once-daily injection of hGH as a lyophilized preparation requiring reconstitution. This treatment regimen has a considerable impact upon patients' lives, and has been show to effect patient compliance. A sustained release formulation of somatotrophic hormones would be desirable, ideally offering improvements in patient comfort and compliance, and product performance.

It would be advantageous to provide a composition for administration of a somatotrophic hormone wherein the release of the somatotrophic hormone is controlled/delayed/sustained resulting in improvements in patient compliance and convenience. Thus, it would be desirable to provide a somatotrophic hormone-containing composition which can be administered less frequently than the known compositions for administration. In the case of hGH, a composition that could be administered every two days, twice weekly, once weekly, once fortnightly, once monthly or even less frequently would be desirable.

The invention provides a solid composition comprising (i) a somatotrophic hormone, (ii) a biodegradable polymer component, and (iii) a release modifier. Unless otherwise stated, this will be referred to hereinafter as the composition of the invention.

Typically, the somatotrophic hormone (i) is present in an amount of from about 1 to about 50% by weight of the composition, such as from about 2 to about 40%, preferably from about 5 to about 30% by weight, for example from about 10 to about 20%.

The biodegradable polymer component (ii) typically is present in an amount of from about 5 to about 98% by weight of the composition, such as from about 25 to about 96.5%, preferably from about 45 to about 93%, for example from about 60 to about 85%.

Typically, the release modifier (iii) is present in an amount of from about 1 to about 45% by weight of the composition, such as from about 1.5 to about 35%, preferably from about 2 to about 25% by weight, for example from about 5 to about 20%.

By the term “somatotrophic hormone”, we mean any hormone that has a stimulating effect on body growth, including human and animal growth hormones. Examples of human and animal growth hormones include bovine and porcine growth hormones, growth hormone releasing hormone and human growth hormone (hGH).

The somatotrophic hormones to be used in the subject invention may be manufactured by recombinant DNA technology. Somatotrophic hormones manufactured in this way are typically isolated and purified as an aqueous solution. In the subject invention, the somatotrophic hormone typically is used in the form of a powder to make the compositions of the invention.

Somatotrophic hormone powders may be formed from somatotrophic hormone solutions using any suitable method known in the art. Suitable methods include, but are not limited to, freeze-drying (lyophilisation), spray drying, air drying, vacuum drying and supercritical fluid technology. Spray drying is preferred.

The somatotrophic hormone can be dried alone or, to improve stability, in the presence of an additive. Suitable additives include, but are not limited to, buffer salts such as phosphate, citrate and acetate buffers; sugars such as sucrose and trehalose; surfactants such as polysorbates; amino acids such as glycine; polyols such as mannitol and sorbitol; and polyethylene glycols. It is preferable to dry the somatotrophic hormone in the presence of an additive.

By the term “somatotrophic hormone powder” we mean a powder consisting of a somatotrophic hormone and optionally an additive. Typically, the additive does not comprise the polymer component (ii) or the release modifier (iii). However, it may be preferable to combine the somatotrophic hormone with at least a portion of the release modifier which is present in the compositions of the invention.

The dry somatotrophic hormone powder preferably comprises at least 40% by weight, more preferably at least 50% and most preferably at least 60% by weight somatotrophic hormone.

The dry somatotrophic hormone powder preferably has a particle size in the range of from 1 nm to 100 μm, more preferably from 1 to 50 μm and most preferably from 1 to 20 μm (e.g. from 1 to 5 μm). More specifically, the mean particle size, expressed as the volume mean diameter (VMD) and measured by a technique such as light microscopy combined with image analysis, lies within these ranges.

A particularly preferred somatotrophic hormone for use in the compositions of the invention is human somatotrophic hormone (hGH), which is also known as somatropin and has a molecular weight of 22 kDa. By “human somatotrophic hormone” or “hGH”, we mean naturally occurring or synthetic somatropin or analogues thereof (e.g. somatrem). hGH typically is used in solid form in the compositions of the invention, preferably as a spray-dried powder.

Any suitable biodegradable polymer(s) may be used in component (ii) of the composition of the invention that is/are suited for introduction into or association with the human or animal body. Preferably the polymer(s) used to make the composition of the invention is/are in the form of a powder.

Preferably a biodegradable polymer is selected from homopolymers, block and random copolymers, polymeric blends and composites of monomers which may be straight chain, (hyper) branched or cross-linked.

Suitable synthetic biodegradable polymers include those disclosed in “Polymeric Biomaterials” ed. Severian Dumitriu, ISBN 0-8247-8969-5, Publ. Marcel Dekker, New York, USA, 1994 (incorporated herein by reference). Examples of types of synthetic biodegradable polymers which may be used in the compositions of the invention are set out below.

Polyesters including poly(lactic acid) (PLA), poly(glycolic acid) (PGA) copolymers of lactic and glycolic acid (PLGA), copolymers of lactic and glycolic acid with poly(ethylene glycol), poly(ε-caprolactone) (PCL), poly (3-hydroxybutyrate) (PHB), poly (p-dioxanone), poly(propylene fumarate).

Modified esters such as poly(ortho esters) including polyol/diketene acetals addition polymers (as described by Heller in: ACS Symposium Series 567, 292-305, 1994, which is incorporated herein by reference) and poly(ether ester) multiblock copolymers such as those based on poly(ethylene glycol) and poly(butylene terephthalate).

Polyan hydrides including poly(sebacic anhydride) (PSA), poly (carboxybiscarboxyphenoxyphenoxyhexane) (PCPP), poly [bis (p-carboxyphenoxy) methane] (PCPM) and copolymers thereof, as described by Tamada and Langer in Journal of Biomaterials Science-Polymer Edition, 3, 315-353, 1992 and by Domb in Chapter 8 of the Handbook of Biodegradable Polymers, ed. Domb A. J. and Wiseman R. M., Harwood Academic Publishers (both of which are incorporated herein by reference).

Poly(amino acids) and poly(pseudo amino acids) including those described by James and Kohn in pages 389-403 of Controlled Drug Delivery Challenges and Strategies, American Chemical Society, Washington D.C. (incorporated herein by reference).

Polyphosphazenes including derivatives of poly[(dichloro)phosphazene], poly[(organo) phosphazenes], polymers described by Schacht in Biotechnology and Bioengineering, 52, 102-108, 1996 (incorporated herein by reference). Azo polymers including those described by Lloyd in International Journal of Pharmaceutics, 106, 255-260, 1994 (incorporated herein by reference).

Natural biodegradable polymers which may be used in component (ii) of the compositions of the invention include starch, cellulose and derivatives thereof including ethylcellulose, methylcellulose, ethylhydroxyethylcellulose, sodium carboxymethylcellulose. Further natural polymers include collagen, gelatin, dextran, alginates, chitin, chitosan and derivatives thereof.

A mixture of one or more of the biodegradable polymers set out above may be used as the biodegradable polymer component. For the avoidance of doubt a mixture of one or more classes of polymers may be used (e.g. a polyester and a polyanhydride) and/or one or more particular polymers in a class.

The biodegradable polymer component currently preferably comprises PCL, PHB, poly(ether ester) multiblock copolymers, PLGA, PLA, or a combination thereof, most preferably PLGA, PLA, or a combination of PLA and PLGA.

PLGA is polylactic-co-glycolic acid). The amount of lactic acid and glycolic acid comonomers present in the PLGA which may be used in the present invention may vary over a wide range. The PLGA may have a molar ratio of lactic acid:glycolic acid of from about 90:10 to about 10:90, such as from about 75:25 to about 25:75, for example about 50:50.

The molecular weight of a polymer is related to its inherent viscosity. The inherent viscosity of the biodegradable polymers that may be used in component (ii) of the compositions of the invention (e.g. PLGA and PLA) typically is from about 0.1 to about 1.5 dl/g, such as from about 0.11 to about 1 or about 0.12 to about 0.5, for example from about 0.15 to about 0.30 or about 0.16 to about 0.24.

In a currently particularly preferred aspect of the invention, the biodegradable polymer component comprises both PLGA and PLA. The ratio (by weight) of PLGA:PLA when they are both present in the biodegradable polymer component typically is from about 95:5 to about 5:95. Preferably, there is about the same or more PLGA present than PLA, for example the weight ratio of PLGA:PLA is from about 90:10 to about 40:60, such as from about 85:15 to about 50:50, for example from about 75:25 to about 60:40.

Without being bound by theory, it is believed that the biodegradable polymer component may help to reduce the “burst release” of the composition of the invention when it is injected into the body. By “burst release”, we mean the amount of somatotrophic hormone, as a percentage of the total amount of somatotrophic hormone in the composition, that is released immediately or substantially immediately (e.g. within about 1 hour) following administration in vivo or dissolution in vitro using standard dissolution tests (e.g. as described herein).

Typically, the burst release of the compositions of the invention is less than about 80%, preferably, less than 70, 60, 50, 40, 30, 20 or 10%.

It is also believed that the biodegradable polymer component helps to control/sustain/delay the release of the somatotrophic hormone following “burst”. In fact, it is thought that the release of somatotrophic hormone following burst in some cases may be too slow using a biodegradable polymer alone. It is believed that the release modifier in the compositions of the invention helps to increase the rate of release of the protein following burst.

Without being bound by theory, it is believed that the release modifier is capable of blending the somatotrophic hormone and the biodegradable polymer component more intimately. Suitable release modifiers include oligomers or polymers with amphiphilic character. Typically, the release modifier has a hydrophilic component and a hydrophobic component. One or more of such release modifiers may be included in the release modifier (iii) of the subject invention.

The release modifier typically has a molecular weight of from about 200 to about 30000 Da or about 250 to about 20000, such as from about 300 to 10000, for example from about 400 to 6000. The release modifier may be a solid (e.g. a powder) or a liquid at room temperature.

Suitable release modifiers include oligomers or polymers of fatty acids, fatty acid esters, hydroxy fatty acid esters, pyrolidones or polyethers, medium and long chain triglycerides, poloxamers, phospholipids, derivatives thereof and mixtures thereof.

Fatty acids which are suitable for use as processing aids include linear and cyclic (preferably linear), saturated and unsaturated fatty acids comprising from 6 to 40, preferably from 9 to 30 and most preferably from 11 to 18 carbon atoms. The saturated fatty acids have the general formula CₙH₂ₙO₂, wherein n is from 7 to 40, preferably from 9 to 30 and most preferably from 11 to 18. The unsaturated fatty acids may have the formula CₙH₂ₙ₋₂O₂, or CₙH₂ₙ₋₄O₂ or CₙH₂ₙ₋₆O₂ wherein n is from 7 to 40, preferably from 9 to 30 and most preferably from 11 to 18. Unsaturated fatty acids with 4 or more double bonds may also be used. Optionally, the fatty acids may be hydroxylated (e.g. 12-hydroxy steric acid). The hydroxy group(s) may be further esterified with another fatty acid (i.e. fatty acid oligomers or polymers). Unsaturated fatty acids may be in the cis- or trans-configurations or mixtures of both configurations may be used.

Examples of preferred fatty acids include stearic acid, oleic acid, myristic acid, caprylic acid and capric acid. Oils containing these and any of the foregoing fatty acids may also be used as the processing aid, e.g. cotton seed oil, sesame oil and olive oil.

Suitable fatty acid derivatives (e.g. esters) include those that can be derived from the fatty acids and hydroxyl fatty acids defined above. Preferred fatty acid esters are mono-esters and di-esters of fatty acids, and derivatives thereof, such as polyethylene glycol (PEG) mono-esters and di-esters of fatty acids. Suitable PEG's include those having from 2 to 200 monomer units, preferably 4 to 100 monomer units, for example 10 to 15 monomer units. Examples include PEG stearate and PEG distearate, each available with varying PEG chain lengths, e.g. polyoxyl 40 stearate (Crodet S40, Croda) and PEG-8 distearate (Lipopeg 4-DS, Adina).

A particularly preferred fatty acid ester for use in the process of the invention is Solutol® HS 15, which is available from BASF. Solutol® consists of polyglycol mono- and di-esters of 12-hydroxystearic acid and of about 30% free polyethylene glycol and is an amphiphilic material having a hydrophilic-lipophilic balance between about 14 and 16.

Further examples of fatty acid derivatives include fatty acids esterified with polyoxyethylene sorbitan compounds, such as the “Tween” compounds (e.g. polyoxyethylene (20) sorbitan monooleate, also known as Tween 80) and fatty acids esterified with sorbitan compounds, such as the “Span” compounds (e.g. sorbitan monooleate, also known as Span 80).

Suitable pyrolidones include 2-pyrolidone and N-methyl-2-pyrrolidone.

Suitable polyethers include those comprising monomers comprising from 2 to 10 carbon atoms, preferably polyethylene glycols (PEGs) and polypropylene glycols (PPG's).

Suitable triglycerides include saturated and unsaturated medium and long chain mono-, di- and tri-glycerides.

Typically, medium chain mono-, di- and tri-glycerides have a formula (CH₂OR₁)(CH₂OR₂)(CH₂OR₃) wherein R₁, R₂ and R₃ are independently H or —C(O)(CH₂)ₙCH₃ (where n=6 to 8), provided that at not all R₁, R₂ and R₃=H. Preferable medium chain mono-, di- and tri-glycerides consist of a mixture of esters of saturated fatty acids mainly of capryilic acid and capric acid e.g. Crodamol GTC/C (Croda), Miglyol 810, Miglyol 812, Neobee M5.

Typically, long chain mono-, di- and tri-glycerides have a formula (CH₂OR₁)(CH₂OR₂)(CH₂OR₃) wherein R₁, R₂ and R₃ are independently H or —C(O)(CH₂)ₘCH₃ (where m=7 to 17), provided that at not all R₁, R₂ and R₃=H. A preferred long chain mono-, di- and tri-glyceride is Witepsol

Poloxamers currently are a particularly preferred group of release modifier. Poloxamers are block copolymers of ethylene oxide and propylene oxide. They have the general formula HO(C₂H₄O)ₐ(C₃H₆O)_{b}(C₂H₄O)ₐH wherein a is typically from 2 to 130 and b is typically from 15 to 67.

Several different types of poloxamer are available commercially, from suppliers such as BASF, and vary with respect to molecular weight and the proportions of ethylene oxide “a” units and propylene oxide “b” units. Poloxamers suitable for use as a release modifier in the subject invention typically have a molecular weight of from 2,500 to 18,000, for example from 7,000 to 15,000 Da. Examples of commercially available poloxamers suitable for use in the subject invention include poloxamer 188, which structurally contains 80 “a” units and 27 “b” units, and has a molecular weight in the range 7680 to 9510 and poloxamer 407 which structurally contains 101 “a” units and 56 “b” units, and has a molecular weight in the range 9840 to 14600 (Handbook of Pharmaceutical Excipients, editor A. H. Kippe, third edition, Pharmaceutical Press, London, UK, 2000, which is incorporated herein by reference).

Additional optional components may also be included in the compositions of the invention. For example, inorganic salts may be added, such as zinc carbonate and magnesium carbonate. In one aspect, such salts are not included in the compositions of the invention.

The composition of the invention typically is in the form of a solid, preferably a powder. It is believed that the combination of components (i), (ii) and (iii) results in a somatotrophic hormone-containing composition having improved particle properties compared to known somatotrophic hormone-containing compositions for subcutaneous administration.

The composition of the invention may be in the form of microparticles, such microparticles preferably have a relatively uniform size. Such microparticles may be referred to hereinafter as microparticles of the invention.

The microparticles typically have a mean particle size expressed as the volume mean diameter (VMD) of from about 10 to about 500 μm, preferably from about 20 to about 200 or 250 μm, more preferably from about 30 to about 150 μm, even more preferably from about 40 to 100 μm, for example from about 50 to about 80 μm. The volume mean diameter of the microparticles can be measured by techniques well known in the art such as laser diffraction.

Typically no more than 10% of the microparticles have a diameter (D_{10%}) less than the lower limit of each of the size ranges quoted above respectively and at least 90% of the particles have a diameter (D_{90%}) that does not exceed the upper limit of each of the size ranges quoted above respectively.

The microparticles of the invention may be characterised by their morphology, which may be determined by analysis of a cross section thereof.

The microparticles of the invention may have a relatively smooth surface and a surface area that is typically lower that that of microparticles produced by supercritical fluid processes of the prior art.

An ideal average surface area (IASA) for the microparticles of the invention can be calculated on the basis of the volume mean diameter (VMD) using the following equation.

IASA=4(pi)r²

Wherein r is the volume mean radius (ie half the VMD)

Of course, this calculation assumes that the microparticles are spheres. Ideally, the microparticles of the invention will be spheres. However, it is unlikely that all of the microparticles produced will be spherical (although they may be substantially spherical). Additionally, although the surface of the microparticles produced by the process of the invention is typically smoother that that of particles produced by previously used methods, not all of the particles will have a perfectly smooth surface.

This means that 4 (pi)r² is the lowest possible surface area for the microparticles of the invention. The microparticles of the invention typically have a surface area which is from about 4 (pi)r² to about 10,000×4 (pi)r², preferably from about 4 (pi)r² to about 1000×4 (pi)r², more preferably from about 4 (pi)r² to about 100×4 (pi)r², for example from about 4 (pi)r² to about 10×4 (pi)r², wherein r is half the VMD.

As noted hereinbefore, it is believed that the combination of components (i), (ii) and (iii) results in a somatotrophic hormone-containing composition in which the components are more intimately blended compared to known somatotrophic hormone-containing compositions for subcutaneous administration. Put another way, the compositions of the inventive are believed to be “true blends” as opposed to the phase-separated blends which are characteristic of known somatotrophic hormone-containing compositions.

By “true blends”, we include the meaning that the compositions are well blended in a single, solvent free step at ambient temperatures resulting in surprisingly good sustained release profiles.

Whether a somatotrophic hormone-containing composition is a true blend or phase-separated blend can be determined by differential scanning calorimetry (DSC). This is explained in more detail below.

The or each biodegradable polymer in component (ii) will have a glass transition temperature (T_{g}), a melting temperature (Tₘ) or both a T_{g} and Tₘ. The or each component that makes up the release modifier (iii) will have a glass transition temperature (T_{g}) or a melting temperature (Tₘ) if it is a solid.

In a true-blended composition, the or each T_{g} of the biodegradable polymer component will tend to merge with the T_{g} of the or each release modifier (to exhibit one T_{g}) as shown by DSC. In contrast, in a phase-separated blend typical of the prior art, the T_{g} of the or each biodegradable polymer component will tend to remain distinct from the or each T_{g} of the release modifier as shown by DSC.

Similarly, if the composition contains a component (ii) having two or more biodegradable polymers each having a T_{g} (and a release modifier having a Tₘ), each T_{g} of the biodegradable polymer component will tend to merge with each other (to exhibit one T_{g}) as shown by DSC. In contrast, each T_{g} of the biodegradable polymer component in a corresponding phase-separated blend will tend to remain distinct from each other as shown by DSC.

If the release modifier has a Tₘ, it will tend to be hidden in a true-blended composition of the invention and evident in a corresponding phase-separated composition, as shown by DSC.

As a consequence of the surprisingly advantageous combination of the components of the composition of the invention, the “true blend” or intimate mixing described above may be achieved by simply mixing the components together.

Accordingly, the invention provides a process for preparing a composition comprising a somatotrophic hormone, the process comprising mixing together (i) a somatotrophic hormone, (ii) a biodegradable polymer component, and (iii) a release modifier to provide a uniform blend. Unless otherwise stated, this will be referred to hereinafter as the process of the invention.

An advantage of the process of the invention is that the processing steps are kept to a minimum, thereby preserving the integrity and biological activity of the somatotrophic hormone.

The mixing step of the process of the invention may be achieved by any suitable means. If the somatotrophic hormone-containing powder is produced by freeze drying, its particle size may be heterogeneous and poorly defined. Therefore, prior to preparing the composition, the somatotrophic hormone powder will preferably undergo a process to produce particles of a well-defined size. Methods for reduction of particle size are well known to those skilled in the art. Preferred methods for reducing the size of the somatotrophic hormone powder include milling. The particle size can be controlled using standard techniques such as sieving.

To minimise somatotrophic hormone degradation, size reduction is preferably performed using low shear forces and/or at a low temperature. There are numerous types of mill available and these are widely described in literature references, such as in Chapter 2, Pharmaceutical Principles of Solid Dosage Forms, J. T. Carstensen, Technomic, Lancaster, Pa., 1993 and Chapter 37, Remington: The Science and Practice of Pharmacy, 20^{th} Edition, Lipincott, Williams and Wilkins, Baltimore, 2000 (both of which are incorporated herein by reference).

For preparing a uniform powder blend on a small scale, a pestle and mortar and/or sieve may be appropriate whereas mechanical mixers are required for larger scale manufacture. There are numerous types of mixer available and these are widely described in the literature, for example Chapter 37, Remington: The Science and Practice of Pharmacy, 20^{th} Edition, Lipincott, Williams and Wilkins, Baltimore, 2000 (incorporated herein by reference).

Alternative processes for preparing the compositions of the invention include spray drying, coacervation and supercritical fluid processes.

In a spray drying process, an aqueous suspension containing the somatotrophic hormone, the biodegradable polymer component and the release modifier is sprayed into a current of hot air which results in rapid evaporation of the water to produce a powder. Further details on spray drying of pharmaceuticals may be found in Broadhead et al., Drug Dev. Ind. Pharm., 18, 1169-1206, 1992.

Preferably, the process of the invention is prepared by a supercritical fluid process.

Thus, the composition of the invention can be obtained by a process which comprises:
a. contacting a mixture of the somatotropic hormone, the polymer or a precursor thereof and a release modifier with a supercritical fluid which is capable of swelling the polymer under temperature and pressure conditions necessary to maintain the fluid in a supercritical state;
b. allowing the supercritical fluid to penetrate and liquefy the polymer, whilst maintaining the temperature and pressure conditions so that the fluid is maintained in a supercritical state;
c. releasing the pressure to precipitate the composition.

This will be referred to hereinafter as the supercritical fluid process of the invention.

In the compositions produced by this method the somatotropic hormone is in substantially unchanged chemical form, and optionally in substantially unchanged physical form.

The process is preferably carried out substantially in the absence of additional carriers or solvents. More preferably, the process is carried out in the absence of additional carriers or solvents.

Without wishing to be bound by theory, it is believed that the absence of additional carriers and solvents helps to ensure that the hormone is substantially unchanged in chemical form and preferably also in physical form during the process of the invention. This means that the hormone retains its activity/performance.

In step b of the supercritical fluid process of the invention the polymer swells. This means that the supercritical fluid dissolves in or permeates the polymer, leading to a depression of the polymer's melting point. This depressions of the polymer's melting point allows it to liquiefy (ie become fluid without dissolving) at a temperature below its melting point. Thus, it is important that the polymer and the supercriticial fluid are selected so that the fluid swells but does not dissolve the polymer. References such as Shine, Chapter 18: Polymers and Supercritical Fluids in Physical Properties of polymers Handbook, 249-256 (passim) (James E Mark ed. 1993), which is incorporated herein by reference, can be used to determine suitable combinations of polymer and supercritical fluid.

In step b the mixture may be blended or mixed, although this is not essential. This may be achieved using methods well known in the art, for example by agitation with associated shear thinning, for example with aeration or fluidising gas flow, stirring or the like, more preferably according to the process of U.S. Pat. No. 5,548,004 (Ferro Corp) the contents of which are incorporated herein by reference.

Step b is typically carried out over a time period of from 1 minute to several hours, for example from 5 minutes to 3 hours, time periods of from about 30 minutes to 2 hours, for example about 1 hour are preferred.

The ingredients used in this process may be combined in any desired order, prior to, or during application of supercritical conditions. For example, prior to step a the polymer and the hormone and optionally the release modifier may be mixed. As a particular, non-limiting example, the hormone may be mixed with the polymer using a freeze drying technique. Using this method can produce a mixture of the hormone and the polymer in which the hormone is distributed on the surface of the polymer.

The (supercrital fluid) process of the invention may be carried out as a batchwise or as a continuous process.

Step c may be carried out using any suitable method known in the art. For example in situ, by depressurising a pressure vessel in which the process is carried out, and simultaneously or otherwise ceasing mixing. Alternatively, the contents of pressure vessel in which the process is conducted may be discharged into a second pressure vessel at lower pressure whereby a homogeneous porous powder of polymer as hereinbefore defined is obtained by known means. Methods which comprise spraying into liquid nitrogen can also be used

Step c can be carried out using techniques for removing a gas, which are similar to spray drying techniques. Apparatus suitable for these techniques and the techniques themselves, are well known.

Step c can be used to facilitate control of the size of particles of the composition. Typically the blended mixture is removed from the mixing chamber (which is under supercritical conditions) into a separate container (which is not under supercritical conditions and may for example be under atmospheric conditions) through a nozzle or like orifice. The size of the aperture of the nozzle or orifice can optionally be controlled to control the size of the particles. Altering the conditions under which the blended material is removed from the supercritical fluid or the rate of removal can also affect that particle size.

In step c, the pressure can be released over a time period of fractions of a second to several days. It is currently preferred to release the pressure rapidly. By rapidly we mean over a period of 5 minutes or less, more preferably 1 minute or less, more preferably a second or less, for example half a second or less.

The supercritical fluid used in the invention can be any fluid which may be brought into a supercritical state. As is known in the art, such fluids may be subjected to conditions of temperature and pressure up to a critical point at which the equilibrium line between liquid and vapour regions disappears. Supercritical fluids are characterised by properties which are both gas like and liquid like. In particular, the fluid density and solubility properties resemble those of liquids, whilst the viscosity, surface tension and fluid diffusion rate in any medium resemble those of a gas, giving gas like penetration of the medium

Supercritical fluids which may be used include carbon dioxide, di-nitrogen oxide, carbon disulphide, aliphatic C₂₋₁₀ hydrocarbons such as ethane, propane, butane, pentane, hexane, ethylene, and halogenated derivatives thereof such as for example carbon tetrafluoride or chloride and carbon monochloride trifluoride, and fluoroform or chloroform, C₆₋₁₀ aromatics such as benzene, toluene and xylene, C₁₋₃ alcohols such as methanol and ethanol, sulphur halides such as sulphur hexafluoride, ammonia, xenon, krypton and the like. Preferably the fluid is carbon dioxide alone or in combination with one or more of the fluids listed above.

Optionally, the supercritical fluid may comprise a co-solvent such as acetone or an alcohol.

Typically these fluids may be brought into supercritical conditions at a temperature of from about 0 to about 300° C. and a pressure of from about 7×10⁵ Nm⁻² to about 1×10⁸ Nm⁻², preferably from about 12×10⁵ Nm⁻² to about 8×10⁷ Nm⁻² (7-1000 bar, preferably 12-800 bar).

It will be appreciated that the choice of fluid will depend on a variety of factors including the nature of the polymer. The nature of the polymer is particularly important in the selection of the supercritical fluid. The fluid must swell the polymer to a sufficient extent so that when the pressure on the mixture is released the fluid will occupy the overwhelming majority of the total volume of the mixture (typically greater than 90% of the total volume). In practical terms, this means that the fluid should have an appropriate combination of high density (ie much greater than the density at atmospheric temperature and pressure) and high solubility in the polymer.

The amount of supercritical fluid used in the process of the invention can vary within wide limits and may depend on factors such as the nature of the polymer and the nature of the reaction vessel.

As used herein, the term “supercritical fluid” should be understood to encompass near supercritical fluids. That is highly compressed fluids that are below the critical temperature point but exhibit many of the same properties as true supercritical fluids. Correspondingly, the term “supercritical state” is considered to encompass near-supercritical state.

Additional components which may be used in the process of the invention include, but are not limited to, initiators, accelerators, hardeners, stabilisers, antioxidants, adhesion promoters, fillers and the like may be incorporated within the polymer. Markers and tags and the like may be incorporated to trace or detect administration or consumption of the composition according to known techniques.

If it is desired to introduce an adhesion promoter into the polymer composition, the promoter may be used to impregnate or coat particles of hormone prior to introduction into the polymer composition, by means of simple mixing, spraying or other known coating techniques, in the presence or absence of a fluid as hereinbefore defined. Preferably coating is performed in conjunction with mixing with fluid as hereinbefore defined. For example, the adhesion promoter may be dissolved in fluid as hereinbefore defined and the solution contacted with the hormone as hereinbefore defined. Alternatively, the adhesion promoter may be introduced into the autoclave during the mixing and/or polymerisation step whereby it attaches to the biologically active material particles in desired manner.

The hormone may be treated prior to or during the incorporation into the polymer with any suitable materials adapted to enhance the performance or mechanical properties thereof. The hormone may, for example, be treated with components such as binders adapted to promote adhesion to the polymer, dispersants to increase dispersion throughout the polymer and prevent aggregate formation, to increase dispersion as a suspension throughout a supercritical fluid, activators to accelerate any biofunctional effect in situ and the like.

Preferred adhesion promoters are soluble in the fluid as hereinbefore defined. This means that any residual promoter that does not bind to the hormone or to the polymer is removed when the microparticles are removed from the supercritical fluid.

The compositions of the invention may be formulated so that they can be administered subcutaneously, intramuscularly, intraperitoneally, nasally, topically and via the pulmonary route (by inhalation). Subcutaneous and intramuscular administration are preferred.

Thus, the invention provides a formulation for subcutaneous, intramuscular, intraperitoneal, nasal, pulmonary and topical administration, the formulation comprising (i) a somatotrophic hormone, (ii) a biodegradable polymer component, (iii) a release modifier and (iv) a pharmaceutically acceptable carrier.

Any pharmaceutically acceptable carrier may be used, depending on the mode of administration. For example, the pharmaceutical carrier may be deionised water or a buffer solution (for example, 3% w/v carboxymethylcellulose, 0.9% w/v sodium chloride in 1 mM phosphate) in which the composition of the invention is suspended. Such a formulation may be administered subcutaneously, intramuscularly or intraperitoneally, preferably subcutaneously or intramuscularly.

The composition may be administered subcutaneously or intramuscularly as a depot. In this formulation, the pharmaceutically acceptable carrier is typically an oil (e.g. sesame oil) a solid or an implant.

The composition may also be administered topically, for example onto a wound to facilitate healing of the wound. In this formulation, the pharmaceutically acceptable carrier may be a cream, gel, paste, spray, suspension. Alternatively, the compositions of the invention may be administered topically as a powder, microparticles or granules without a pharmaceutically acceptable carrier.

The compositions of the invention may be used to promote growth of a human or animal body.

The compositions of the invention may be administered to animals, e.g. livestock, to promote growth, e.g. to increase milk or meat production.

Human growth hormone may be administered to a human to treat and/or prevent growth retardation, growth hormone deficiency or the HIV-related wasting and cachexia (e.g. HIV-associated adipose redistribution syndrome (HARS)).

The growth retardation may be caused by insufficient somatotrophic hormone deficiency, Turner's syndrome or chronic renal insufficiency.

The invention will now be illustrated by the following non-limiting examples.

### EXAMPLE 1

hGH, which is obtainable from Hospira (Adelaide), was in the form of an ammonium bicarbonate solution and was spray dried (as described in Maa et al, J. Pharm. Sci., no. 2, page 152 (1998), incorporated herein by reference) prior to combination with the biodegradable polymers and release modifier, as follows.

| | Component/% b/w | | | | |
|---|---|---|---|---|---|
| | Spray dried | PLGA | PLA | Poloxamer | Poloxamer |
| Composition | hGH/% b/w | (RG502H) | (R202H) | 188 | 407 |
| 1 (PLGA:PLA = 50:50) | 10 | 45 | 45 | — | — |
| 2 (PLGA:PLA = 65:35) | 10 | 45 | 45 | — | — |
| 3 (PLGA:PLA = 80:20) | 10 | 45 | 45 | — | — |
| 4 (PLGA:PLA = 90:10) | 10 | 45 | 45 | — | — |
| 5 (PLGA:PLA = 85:15) | 10 | 68.85 | 12.15 | 9 | — |
| 6 (PLGA:PLA = 90:10) | 10 | 72.9 | 8.1 | — | 9 |

The PLGA (RG502H) was obtained from Boehringer Ingelheim and had an inherent viscosity of 0.16-0.24 dL/g and a lactic acid:glycolic acid ratio of 50:50. The PLA (R202H) was obtained from Boehringer Ingelheim an inherent viscosity of 0.16-0.24 dL/g.

The spray dried hGH and excipients were placed in a high pressure mixing chamber and the polymer liquefied using scCO₂ (>76 bar/32° C.) and mixed for 1 hour. Spraying the mixture through a nozzle yielded PLGA microparticles containing hGH. The encapsulation efficiency of the formulated drug was found to be 98±3%, with no visible aggregation of hGH.

### EXAMPLE 2

In vitro release was assessed by weighing triplicate samples of each composition into eppendorf tubes, and suspending them in a release buffer consisting of 10 mM HEPES pH 7.4, 100 mM NaCl, 0.1% Tween 20, and 0.1% NaN₃. The samples were put onto a rotator mixer set to 10 rpm and incubated at 37° C. At various time points the release medium was sampled and replaced, and assayed for hGH content using the SEC method described in the European Pharmacopoeia (incorporated herein by reference).

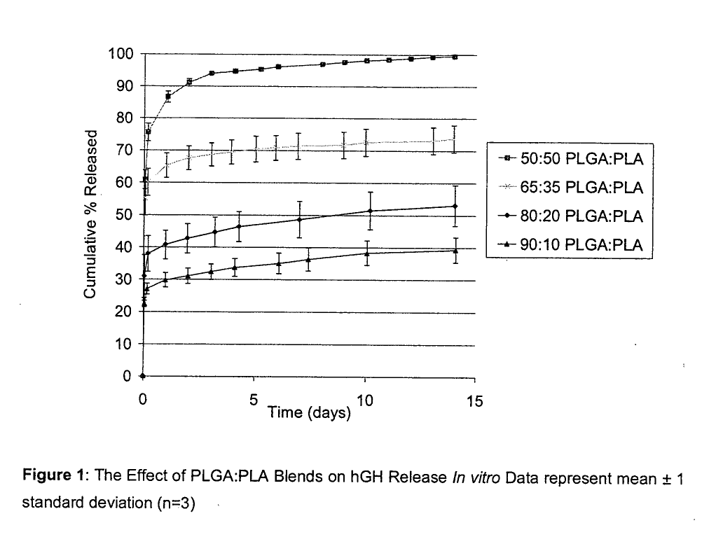

The results for comparative compositions 1 to 4 are set shown in FIG. 1. By increasing the PLGA content, burst release was reduced, but the subsequent release rate was slower than desired.

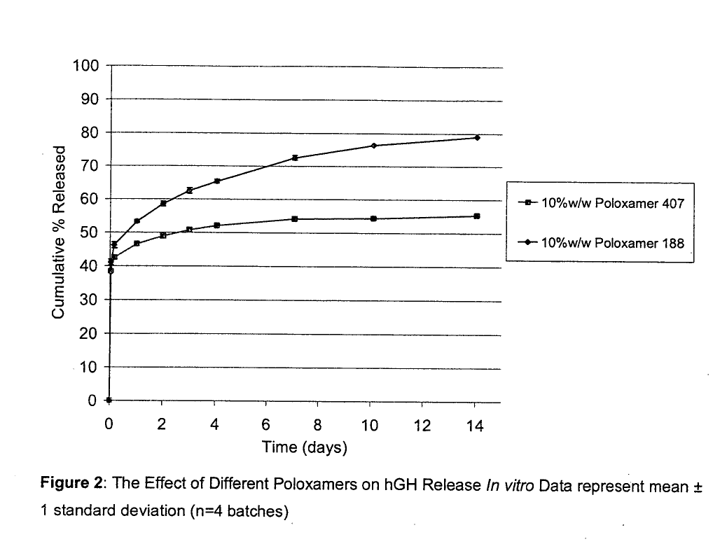

The results for compositions of the invention 5 and 6 are shown in FIG. 2. By incorporating different poloxamers the burst release could be controlled, and the subsequent release rate modified.

### EXAMPLE 3

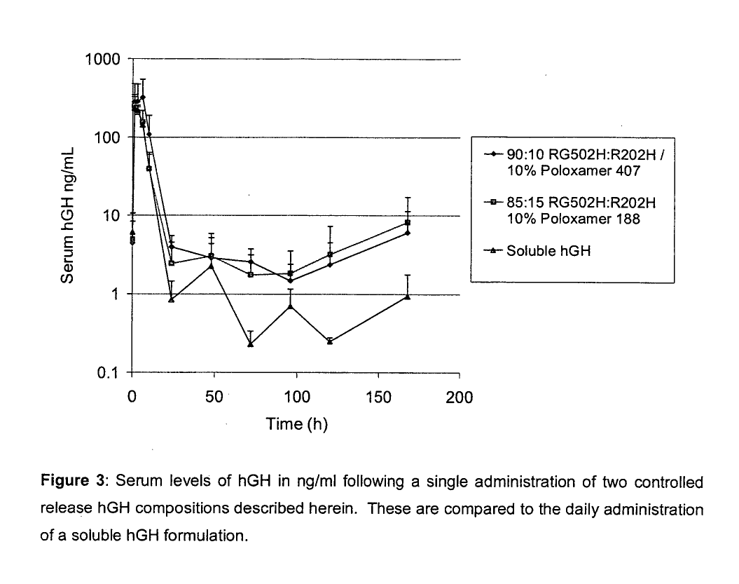

Compositions 5 and 6 described in Example 1 were suspended in a re-suspension buffer consisting of 0.5% w/v carboxymethylcellulose, 0.9% w/v sodium chloride in 1 mM phosphate buffer and were administered once in vivo to two groups of Cynomolgus monkeys by subcutaneous administration and compared to 7 daily single doses of immediate release hGH (the spray dried hGH dissolved in the re-suspension vehicle described above). hGH levels in the serum were determined by enzyme linked immunosorbant assay (ELISA) at daily intervals for seven days after administration. The results are illustrated in FIG. 3.

Initial release of the compositions of the invention was comparable to the immediate release soluble formulation, and ongoing serum concentrations were elevated when compared to the daily administration.

1. A solid composition comprising (i) a somatotrophic hormone; (ii) a biodegradable polymer component; and (iii) a release modifier.

2. A composition according to claim 1, wherein the somatotrophic hormone comprises from about 1 to about 50% by weight of the composition.

3. A composition according to claim 1, wherein the biodegradable polymer component comprises from about 5 to about 98% by weight of the composition.

4. A composition according to claim 1, wherein the release modifier comprises from about 1 to about 45% by weight of the composition.

5. A composition according to claim 1, wherein the somatotrophic hormone is human growth hormone (hGH).

6. A composition according to claim 1, wherein the composition is in the form of particles having a volume mean diameter (VMD) of from about 10 to about 500 μm, preferably from about 40 to about 100 μm.

7. A composition according to claim 1, wherein the biodegradable polymer component comprises (i) a synthetic biodegradable polymer selected from a polyester, a modified polyester, a polyanhydride, a poly(amino acid), a polyphosphazene, mixtures thereof and derivatives thereof; and/or (ii) a natural biodegradable polymer.

8. A composition according to claim 1, wherein the biodegradable polymer component comprises a polyester.

9. A composition according to claim 8, wherein the biodegradable polymer component comprises poly(lactic acid) (PLA), poly(lactic-co-glycolic acid) (PLGA) or a mixture thereof.

10. A composition according to claim 9, wherein the biodegradable polymer component comprises PLGA and PLA in a ratio by weight of from about 95:5 to about 5:95 PLGA:PLA.

11. A composition according to claim 1, wherein the release modifier is selected from oligomers or polymers of fatty acids, fatty acid esters, hydroxy fatty acid esters, pyrolidones or polyethers, medium and long chain triglycerides, poloxamers, phospholipids, derivatives thereof and mixtures thereof.

12. A composition according to claim 11, wherein the release modifier comprises a poloxamer.

13. A composition according to claim 12, wherein the release modifier comprises poloxamer 188, poloxamer 407 or a mixture thereof.

14. A composition according to claim 1, wherein the solid composition comprises microparticles.

15. A composition according to claim 14, wherein the microparticles have a surface area which is from about 4 (pi)r² to about 1000×4 (pi)r², wherein r is half the volume mean diameter.

16. A composition according to claim 1, wherein the composition is a true blend, as determined by differential scanning calorimetry.

17. A process for preparing a composition comprising a somatotrophic hormone, the process comprising mixing together (i) a somatotrophic hormone, (ii) a biodegradable polymer component, and (iii) a release modifier to provide a uniform blend.

18. A composition according to claim 18, wherein the hGH is in the form of a spray dried powder.

19. A process according to claim 17, wherein the mixing comprises a supercritical fluid process.

20. A process according to claim 19, which comprises:
a) contacting a mixture of the somatotropic hormone, the polymer or a precursor thereof and a release modifier with a supercritical fluid which is capable of swelling the polymer under temperature and pressure conditions necessary to maintain the fluid in a supercritical state;
b) allowing the supercritical fluid to penetrate and liquefy the polymer, whilst maintaining the temperature and pressure conditions so that the fluid is maintained in a supercritical state;
c) releasing the pressure to precipitate the composition.

21. A formulation comprising a composition as defined in claim 1 in a pharmaceutically acceptable carrier and formulated for subcutaneous, intramuscular, intraperitoneal or topical administration.

22. (canceled)

23. A method of promoting growth of a human or animal body, treating and/or preventing growth retardation, growth hormone deficiency or HIV-relating wasting and cachexia, the method comprising administering a composition according to claim 1 to a human or animal patient.

24-25. (canceled)

26. The method, according to claim 23, wherein the growth retardation is caused by insufficient growth hormone deficiency, Turner's syndrome or chronic renal insufficiency.

27. The method according to claim 23, wherein the HIV-relating wasting and cachexia is HIV-associated adipose redistribution syndrome (HARS).

28-33. (canceled)
